(19) United States
(12) Patent Application Publication
(10) Pub. No.: US 2012/0142767 A1
(43) Pub. Date: Jun. 7, 2012

(54) PINE PCO ESTER COSMETIC COMPOSITION

(75) Inventor: Nicolas Huguet, Leon (FR)

(73) Assignee: ACTION PIN, Dax (FR)

(21) Appl. No.: 13/375,064

(22) PCT Filed: Jun. 1, 2010
(86) PCT No.: PCT/FR10/51054

§ 371 Date: Feb. 20, 2012

(30) Foreign Application Priority Data
Jun. 2, 2009 (FR) 0953620

(51) Int. Cl. A61K 8/92; A61Q 19/08

(52) U.S. Cl. 514/456

(57) ABSTRACT

The invention relates to a cosmetic composition for repairing and optionally preventing the effects of skin ageing, based on an extract of Pinus pinaster bark, the procyanidolic oligomers (PCOs) of which are esterified with a fatty acid. These compositions are particularly useful for increasing collagen synthesis, for soothing the skin, or further for protecting the skin against free radicals.

The invention relates to a cosmetic composition for repairing and possibly preventing effects of ageing on the skin, based on an extract of pinus pinaster bark, the procyanidolic oligomers (PCOs) of which are esterified with a fatty acid. These compositions are notably useful for increasing collagen synthesis, for soothing the skin, or further for protecting the skin against free radicals.

Procyanidolic oligomers (PCOs) also called proantho-cyanidins or proanthocyanidic oligomers, are very widespread natural molecules in nature. Indeed, they are found in vegetables, fruit, cereals but also in grape pips and pine bark. The nutritional and physiological advantage of these molecules is related to their anti-oxidant properties and to their benefit on the cardiovascular system.

PCOs belong to the chemical family of polyphenols and more particularly to the class of flavonoids, compounds recognized for their anti-oxidant properties. PCOs are formed by one or more recurrent monomer units of flavan-3-ol, these units being independently selected from catechin or epicatechin or epicatecin gallate. These units are generally repeated two to four times, the most common PCOs generally being in the dimer form.

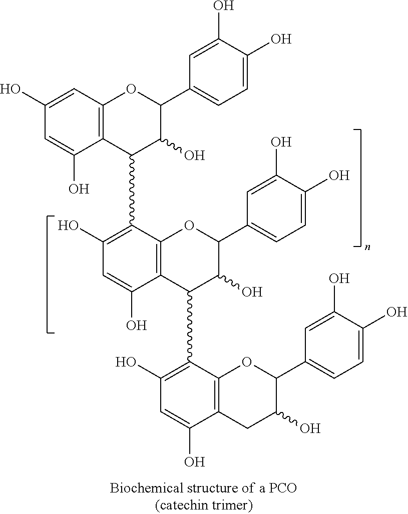

However, the phenolic functions of PCOs make these compounds particularly sensitive to different degradation factors such as light, air, and acid or basic pHs. This instability in the majority of the cases is an obstacle to exploiting their beneficial effects. In order to stabilize these compounds, esterification of the polyphenolic oligomer extracts by derivatives of fatty acids with long hydrocarbon chains (FR 2 723 943) has notably been proposed.

The inventors have now shown that a pine bark extract, the procyanidolic oligomers (PCOs) of which are esterified with a fatty acid, has particularly interesting cosmetic properties. It was thus shown that PCO esters extracted from pine bark gave the possibility of increasing the synthesis of collagen by the fibroblasts of the human dermis, and therefore of repairing the effects of skin ageing. It was also shown that these PCO esters further had anti-inflammatory and antiradical properties, making them particularly useful for soothing the skin and preventing the effects of skin ageing.

The object of the present invention is thus a cosmetic composition for repairing the effects of skin ageing comprising:
a) procyanidolic oligomers (PCOs) extracted from pine barks, said oligomers being esterified with at least one fatty acid; and
b) a cosmetically acceptable carrier.

According to a preferred embodiment, the cosmetic compositions according to the invention comprise:
a) a pine bark extract, the procyanidolic oligomers (PCOs) of which are esterified with at least one fatty acid, and
b) a cosmetically acceptable carrier.

By “cosmetically acceptable carrier” is meant a carrier adapted for use in contact with human and animal cells, in particular cells of the epidermis, without any undue toxicity, irritation, allergic reaction and the like, and proportioned at a advantage/reasonable risk ratio.

The extracts of pine bark, notably of maritime pine, contain a great variety of procyanidolic oligomers, notably including catechin monomers, and of taxifoliol as well as procyanidolic oligomers with 2 to 5 units of flavan-3-ol, the monomers and dimers being present in majority.

The composition of the pine bark extracts is differentiated in a qualitative and/or quantitative way from that of the other extracts of plant origin, in particular extracts of grape pips, which are notably found commercially under the name of VITAFLAVAN®. More specifically, the procyanidolic oligomers extracted from pine bark notably comprise taxifoliol, taxifoliol glucoside, ferulate glucoside and different phenolic acids, which are not present in the grape pip extracts. Unlike the latter, they do not comprise epicatechin or epicatechin gallate on the other hand. The PCOs generally represent about 70% by weight of the total weight of the pine bark extract.

Extracts of maritime pin bark are available commercially (OLIGOPIN®, DRT), they may also be obtained by a conventional extraction method as the one described in patent FR 998 508.

The pine bark extract may notably be an extract of a French maritime pine (Pinus pinaster), such as pine from the Landes.

According to a preferred embodiment of the invention, the procyanidolic oligomers (PCOs) contained in the maritime pine bark extracts are esterified with a fatty acid according to the method described in patent application FR 2 723 943. More particularly, the PCOs are esterified according to the method comprising the steps:
a) contacting the maritime pine bark extract in a liquid medium non-solvent for said extract but solvent for the esters to be obtained, so as to obtain a suspension;
b) adding to said suspension at least one low melting point aliphatic tertiary amine, in the presence of a catalytic amount of at least one organic base other than pyridine, and
c) introducing into this mixture at least one fatty acid chloride, the reaction mixture being stirred at a temperature below 40° C. and then concentrated by evaporation in order to obtain an extract in which the PCOs are esterified.

Preferably, the PCOs are esterified by saturated fatty acids, notably palmitic acid and stearic acid, more preferentially palmitic acid.

The application of a cosmetic composition according to the invention is carried out via a topical route.

The composition is more particularly intended for treating the skin and may appear as an ointment, cream, oil, milk, a pomade, powder, impregnated wad, solution, gel, spray, lotion, suspension, soap.

The compositions according to the invention may be cosmetic compositions in the form of an oil-in-water or a water-in-oil emulsion or multiple emulsions, as a micro-emulsion, hydroalcoholic gel, cream, oil, hydroalcoholic lotion.

Preferentially, the cosmetic compositions according to the invention may comprise from 0.1 to 2.5% of said esterified extract expressed as a weight relatively to the total weight of the composition, preferably from 0.2 to 1.0%.

The cosmetic compositions according to the invention are particularly useful for increasing synthesis of collagen, notably by fibroblasts of the human dermis. They are further advantageously useful for soothing the skin and/or for protecting the skin against free radicals. They are thus particularly useful for preventing the effects of skin ageing, notably photo-ageing.

The object of the invention is also the use of a cosmetic composition comprising:
a) procyanidolic oligomers (PCOs) extracted from pine bark, said oligomers being esterified with at least one fatty acid; and
b) a cosmetic acceptable carrier,
in order to repair the effects of skin ageing.

The present invention also relates to a cosmetic treatment method, comprising the application, notably topical application, of a cosmetic composition as defined above.

Thus, the present invention relates to a cosmetic treatment method, for repairing the effects of skin ageing, comprising the application of a cosmetic composition comprising procyanidolic oligomers (PCOs) extracted from pine barks, said oligomers being esterified with at least one fatty acid; and a cosmetically acceptable carrier.

The present invention also relates to a cosmetic treatment method, for repairing the effects of skin ageing, comprising the application of a cosmetic composition, comprising an extract of pine bark, the procyanidolic oligomers (PCOs) of which are esterified with at least one fatty acid, and a cosmetic acceptable carrier.

The examples which follow illustrate the invention without however limiting it.

### EXAMPLES

### Example 1

Preparation of an Extract of Maritime Pine Bark Extract Containing Esterified PCOs

The pine bark extract (Oligopin®) is esterified with palmitic acid according to the method described in patent application FR 2 723 943.

In the following examples, the terms of “pine PCO esters” refer to the esterified pine bark extract according to the present Example 1.

### Example 2

Study of the Cytotoxic Activity of Pine PCO Esters on a Monolayer of Cells in Culture

1. Goal of the Study

The purpose of the study is to evaluate by an in vitro test, the capacity of pine PCO esters prepared according to Example 1 of inducing cytotoxic effects on a monolayer of human fibroblasts.

2. Principle of the Study

The goal of the study is to evaluate the capacity of pine PCO esters of inducing cytotoxic effects on a primary culture of normal human fibroblasts.

After applying the product on the cells for 24 hours, cell viability is evaluated by measuring the activity of the mitochondrial succinate dehydrogenase of living cells. This enzyme transforms MTT (3(4,5-diméthylthiazol-2-yl)-2,5-diphenyltetrazolium bromide) into blue formazan crystals. After dissolution of these crystals, a spectrophotometric read-out is performed. The measured absorbancies are proportional to the number of living cells.

3. Test System

The cells used are fibroblast from human skin tissue (Caucasian child foreskin) in passage 1.

4. Course of the Study

Reference Elements:

The following controls are included in each analysis:

Negative control: complete culture medium

Positive control: SDS

Definition of the Series:

Six dilutions of each sample, prepared in an culture medium with 0.005% of between 60 and 0.05% of cotton oil, are tested in an amount of 5 test wells per concentration. The reference elements are tested on 5 wells.

5. Expression and Interpretation of Results

The absorbance is measured at 540 nm. The results are expressed as a mortality percentage compared with the negative control.

$$\% of mortality = \frac{negative control abs . - sample abs .}{negative control abs .} \times 100$$

6. Results

| | | UA MTT (N = 8) | |
|---|---|---|---|
| | | Standard deviation | Mortality % |
| | Negative control | 0.245 | 0% |
| | (solvent) | 0.016 | |
| | Positive control | 0.065 | 73% |
| | | 0.011 | |
| Pine PCO esters | 1,000 μg/ml | 0.262 | 0% |
| | | 0.009 | |
| | 500 μg/ml | 0.237 | 4% |
| | | 0.034 | |
| | 100 μg/ml | 0.224 | 9% |
| | | 0.023 | |
| | 50 μg/ml | 0.227 | 8% |
| | | 0.019 | |
| | 10 μg/ml | 0.272 | 0% |
| | | 0.026 | |
| | 1 μg/ml | 0.221 | 10% |
| | | 0.027 | |

The positive control has a mortality of more than 30%, this validates the test.

7. Conclusion

Under the retained experimental conditions, the pine PCO esters according to Example 1 do not have any significant cytotoxic effect. For all the tests of efficiency, the maximum concentration used was 1,000 μg/mL.

### Example 3

Screening Study of the Anti-Inflammatory and Anti-Radical Potential

1. Principle of the Study

A human epidermis is reconstructed in vitro in culture inserts.

A paper filter disc on which the substance to be tested (preventive treatment) is deposited on the “stratum corneum”. After 24 hours, it is irradiated by UVBs and then the substance to be tested (curative treatment) is again applied.

After 24 hours, it is proceeded with:

a study of cytoxicity (MTT),

an assay of IL-1α, a pro-inflammatory mediator in the culture medium,

an assay of MDA (malonaldehyde) (a terminal product formed during the lipid peroxidation process induced by the formation of radical species, or species activated by oxygen).

2. Identification of the Substance to be Tested

Esterified PCO according to Example 1 in a 1% (wlw) solution in solvent E (50% isopropanol-50% isopropyl myristate in w/w)

3. Procedure

3.1. Preparation of the Reactive System: Reconstructed Human Epidermal Layers:

Culture of human keratinocytes at the 3^{rd} passage free of mycoplasms

The keratinocytes sites are sown at a density of 100×10³ cells/cm² in inserts, the bottom of which consists of a polycarbonate membrane (porosity 0.4 μm-1 cm²).

The cells are incubated at 37° C. in a humid atmosphere containing 5% (v/v) CO₂ for 24 hours, in an “immersed situation” in the culture medium: MCDB/153 supplemented with EGF (5 ng/mL), insulin (5 μg/mL), hydrocortisone (0.5 μg/mL), BPE (70 μg/mL).

The culture medium is replaced with the same culture medium supplemented with one mM of Ca⁺⁺ in order to induce stratification of the monolayer in the “immersed situation” for 6 days. The culture medium is changed every three days.

The epidermal layer is put into an “emerged situation” on the 6^{th} day, i.e. it is only fed via the basal cells, the stratum corneum being exposed to air. The culture medium is changed every three days.

3.2. Preparation of the Substance to be Tested:

On the 14^{th} day of incubation, the culture medium is changed and replaced with the same culture medium supplemented with 1% antibiotics (10,000 U penicillin-10,000 μg/mL streptomycin-25 μg/mL amphotericin), 20 μL of the substance to be tested (three non-cytotoxic concentrations) or 20 μL of negative controls (culture medium) or solvent controls are applied under sterile conditions, on a paper filter disk (Whatman n°3 MMChr) covering the surface of the reconstructed epidermis, the side onto which the substance to be tested and the controls in contact with the <<stratum corneum>> have been deposited.

3.3. Definition of the Series:

The following series are applied in triplicate:

3 series of substance to be tested (5, 50 and 500 μg/mL) each consisting of 3 epidermises (to be irradiated).

1 series of absolute negative control (not irradiated),

1 series of absolute negative control (to be irradiated),

1 series of solvent control (not irradiated),

1 series of solvent control (to be irradiated),

1 series of positive control 1 (vitamin E:200 μg/mL) (to be irradiated),

1 series of positive control 2 (indomethacin:10 μg/mL) (to be irradiated),

The first eight series are put away for incubation for 24 hours at 37° C. in a humid atmosphere containing 5% (v/v) of CO₂ (preventive treatment for the treated series).

The ninth series, corresponding to the positive control 2 (indomethacin), is formed by depositing 20 μL of a 10 μg/mL indomethacin solution on a paper filter disk (Whatman n°3 MMChr) deposited in contact with the reconstructed epidermis and incubated for 3 hours at 37° C., 21 hours after applying the preceding series.

Next, the paper filter disks are carefully withdrawn and all the series are irradiated with UVBs (0.6 J/cm²) (Spectroline model X-15N/F, λ=312 nm) except for the series corresponding to the absolute negative control and to the solvent control.

The curative treatment is then applied as described above for the preventive treatment over a period of 24 hours, except for the positive control series 2 (indomethacin).

3.4. Estimation of the Cell Population (Test with MTT*):

The MTT test is carried out according to the conditions described in the publications: Hausen M. B, Nielsen S. E. and Berg K. Re-examination and further development of a precise and rapid dye method for measuring cell growth/cell kill. J. Immunol. Methods. 1989, 119, 203; and Mosmann T., Rapid colorimetric assay for cellular growth and survival: Application to proliferation and cytotoxic assays, J. Immunol. Methods. 1983, 65, 55-63.

At the end of the selected incubation time, the Whatman paper disk is carefully picked up, the <<stratum corneum>> is rinsed and the porous membrane bearing the epidermal layer is detached from the insert with a bistoury. The culture medium are used for assaying IL-1α and MDA.

The epidermal layer is:

rinsed with PBS (twice),

incubated for 3 hours, at 37° C., in darkness, in the presence of 2 mL of MTT (3-{4,5-dimethylthiazol-2-yl}-2,5-diphenyltetrazolium bromide) at 0.5 mg/mL of culture medium,

rinsed with PBS (twice).

The violet formazan crystals produced by cleavage of MTT by mitochondrial enzymes are dissolved in 2 mL of DMSO with mild stirring for one hour at room temperature.

For each layer, 200 μL of the obtained solution are pipeted into an 96 well plate for carrying out spectrometry with a plate reader (FL600—BIO-TEK) at λ=540 nm.

3.5. Assay of IL-1α:

The assay of IL-1α is carried out in the sampled culture media (n=3), diluted to 1/10^{th} and to 1/20^{th} according to the procedure described in the assay kit.

3.6. Assay of MDA:

Preparation of the MDA Range Standard:

a 10 μM malonaldehyde bi[dimethylacetal] mother solution in 1% (v/v) sulfuric acid (2 hours at room temperature),

dilutions are carried out between 0.1 and 10 μM (0.1-1-2.5-5 and 10 μM).

500 μL of culture medium (or 500 μL of a standard range) are energetically mixed in a vortex mixer, with 1 mL of a <<TCA, TBA, HCl>> solution (0.15% (w/v) of TCA trichloroacetic acid, 0.375% (w/v) of TBA (2-thiobarbituric acid), 0.25 M of HCl) (heated for 15 minutes at 100° C. in order to dissolve the TBA, and then left to cool).

The mixture is incubated for 15 minutes at 100° C. and the OD is measured at λ=535 nm against a blank formed with the <<TCA, TBA, HCl>> solution.

4. Results

TABLE 1

| | Concentration | | | |
|---|---|---|---|---|
| Test substance | (μg/mL) | AU* + (MTT) | II-1α(pg/mL) | II-1α(pg/AU MTT) |
| 1% (w/w) esterified PCO | 5 | 0.350 | 204 | 582 |
| solution in solvent E (50% | | 0.260 | 237 | 913 |
| isopropanol-50% isopropyl | | 0.265 | 409 | 1541 |
| myristate in w/w) + | | 0.292 ± 0.051 | 283 ± 90 | 1012 ± 398 |
| UVB | 50 | 0.220 | 337 | 1532 |
| | | 0.361 | 175 | 486 |
| | | 0.306 | 245 | 803 |
| | | 0.295 ± 0.071 | 252 ± 66 | 940 ± 438 |
| | 500 | 0.334 | 271 | 810 |
| | | 0.296 | 256 | 866 |
| | | 0.287 | 193 | 673 |
| | | 0.306 ± 0.025 | 240 ± 34 | 783 ± 81 |

| | | | |
|---|---|---|---|
| Solvent control + | 0.259 | 414 | 1601 |
| UVB | 0.153 | 169 | 1103 |
| | 0.244 | 333 | 1368 |
| | 0.218 ± 0.057 | 305 ± 102 | 1357 ± 203 |
| Negative control + | 0.264 | 350 | 1327 |
| UVB | 0.279 | 191 | 684 |
| | 0.165 | 174 | 1056 |
| | 0.236 + 0.062 | 238 ± 79 | 1022 ± 264 |
| Positive control | 0.214 | 302 | 1414 |
| Indomethacin + UVB | 0.288 | 141 | 490 |
| | 0.232 | 130 | 560 |
| | 0.245 ± 0.039 | 191 ± 79 | 821 ± 420 |
| Solvent control | 0.388 | 59 | 153 |
| | 0.390 | 49 | 127 |
| | 0.323 | 34 | 105 |
| | 0.367 ± 0.038 | 48 ± 10 | 128 ± 19 |
| Absolute negative control | 0.371 | 47 | 127 |
| | 0.319 | 68 | 212 |
| | 0.431 | 67 | 156 |
| | 0.374 ± 0.056 | 61 ± 10 | 165 ± 35 |

*AU: arbitrary units

TABLE 2

| | Con- | | | |
|---|---|---|---|---|
| | cen- | | | |
| | tra- | | | |
| | tion | | | |
| Test | (μg/ | | | MDA (μM/AU |
| substance | mL) | AU* + (MTT) | MDA (μM) | MTT) |
| 1% (w/w) | 5 | 0.350 | 0.529 | 1.510 |
| esterified PCO | | 0.260 | 0.518 | 1.994 |
| solution in | | 0.265 | 0.533 | 2.010 |
| solvent E (50% | | 0.292 ± 0.051 | 0.527 ± 0.008 | 1.838 ± 0.284 |
| isopropanol- | 50 | 0.220 | 0.419 | 1.906 |
| 50% isopropyl | | 0.361 | 0.479 | 1.329 |
| myristate in | | 0.306 | 0.481 | 1.573 |
| w/w) + UVB | | 0.295 ± 0.071 | 0.460 ± 0.035 | 1.603 ± 0.290 |
| | 500 | 0.334 | 0.495 | 1.480 |
| | | 0.296 | 0.609 | 2.062 |
| | | 0.287 | 0.534 | 1.860 |
| | | 0.306 ± 0.025 | 0.546 ± 0.058 | 1.801 ± .296 |

| | | | |
|---|---|---|---|
| Solvent control + | 0.259 | 0.508 | 1.966 |
| UVB | 0.153 | 0.643 | 4.198 |
| | 0.244 | 0.548 | 2.248 |
| | 0.218 ± 0.057 | 0.566 ± 0.069 | 2.804 ± 1.215 |
| Negative control + | 0.264 | 0.557 | 2.110 |
| UVB | 0.279 | 0.526 | 1.887 |
| | 0.165 | 0.554 | 3.357 |
| | 0.236 ± 0.062 | 0.546 ± 0.017 | 2.451 ± 0.792 |
| Positive control | 0.246 | 0.422 | 1.715 |
| Vitamin E + UVB | 0.369 | 0.423 | 1.147 |
| | 0.356 | 0.484 | 1.359 |
| | 0.324 ± 0.067 | 0.443 ± 0.035 | 1.407 ± 0.287 |
| Solvent control | 0.388 | 0.354 | 0.912 |
| | 0.390 | 0.291 | 0.747 |
| | 0.323 | 0.311 | 0.961 |
| | 0.367 ± 0.038 | 0.318 ± 0.032 | 0.873 ± 0.112 |
| Absolute negative | 0.371 | 0.329 | 0.888 |
| control | 0.319 | 0.285 | 0.893 |
| | 0.431 | 0.383 | 0.888 |
| | 0.374 ± 0.056 | 0.332 ± 0.049 | 0.890 ± 0.003 |

*AU: arbitrary units

4.1. Cytotoxicity

Irradiated absolute negative control: a 37% reduction (P<0.05) of the OD (AU MTT) is observed with respect to the absolute negative control, which corresponds to the cytotoxic effect generally obtained for the irradiation dose used (0.6 J/cm²) and validates the irradiation.

Irradiated absolute negative control, treated with indomethacin for 3 hours before irradiation: there is no protective effect of indomethacin as regards cytoxicity.

Irradiated negative control, treated with vitamin E, 24 hours before irradiation: a protective effect on vitamin E is observed since the cytotoxic effect is only 13% (NS), i.e. about one third of the one observed in the absence of vitamin E.

Solvent control: it did not significantly change cell proliferation.

Irradiated solvent control: it is not significantly different from the irradiated absolute negative control.

Test substance: a protective effect is observed. For 500 μg/mL, the cytotoxic effect is 17% (NS).

4.2. Study of the IL-1α Assay (Table 1):

Irradiated absolute negative control: The IL-1α level in the culture medium is very large: +290% (P<0.02) with respect to the absolute negative control. This result validates the reactive system which appears to be particularly reactive.

Irradiated absolute negative control treated with indomethacin 3 hours before irradiation: indomethacin used in prevention partly inhibits (−20%) the synthesis and secretion of IL-1α in the culture medium. Although this effect is not statistically significant, it corresponds to the usually obtained values.

Solvent control: it is not significantly different from the absolute negative control.

Irradiated solvent control: like for the irradiated absolute negative control, the IL-1α level is strongly increased. This increase is not significantly different from the one observed for the irradiated absolute negative control.

Test substance (pine PCO esters): a reduction in the IL-1α level in the culture medium is observed; this effect is particularly clear for the strongest concentration (500 μg/mL): −41% (P<0.02) with respect to the irradiated solvent, which represents twice of the inhibition induced by indomethacin.

4.3. Study of the MDA (Table 2):

Irradiated absolute negative control: the MDA level is increased by 175% (P<0.02) with respect to the non-irradiated absolute negative control, which validates the reactive system.

Irradiated absolute negative control, treated with vitamin E before and after irradiation: the increase in the MDA level is only 58% (P<0.05), which represents the third of the level observed for the non-treated irradiated absolute negative control.

Non-irradiated solvent control: there is no significant effect on the MDA level with respect to the absolute negative control.

Irradiated solvent control: there is no significant effect on the MDA level with respect to the irradiated absolute negative control.

Test substance (pine PCO esters): a reduction in the MDA level is observed in the culture medium for all the investigated concentrations. For 50 μg/mL, a 43% inhibition is observed with respect to the solvent control irradiated with UVBs.

5. Conclusion

For the investigated non-cytotoxic concentrations, 5, 50 and 500 μg/mL, the test substance, the esterified PCO according to Example 1, in solution at 1% (w/w) in the solvent E (50% isopropanol-50% isopropyl myristate in w/w):

protects the irradiated epidermis from cytoxicity induced by UVBs;

reduces the level of IL-1α secreted in the culture medium after irradiation by UVBs: −42% with respect to the irradiated solvent control for 500 μg/mL;

reduces the MDA levels secreted in the culture medium: 35% to 45%.

These <<screening>> results show anti-inflammatory and even antiradical activity.

The positive and negative controls validated the experiment.

### Example 4

Study of the Effect of a Test Element (Pine PCO Esters from Example 1) on the Synthesis of Collagen by Human Fibroblasts

1. Goal of the Study

The purpose of this study is to evaluate by an in vitro test, the effect of pine PCO esters according to Example 1 on the synthesis of collagen by a culture of human primary fibroblasts.

2. Principle of the Study

Fibroblasts are the main cells of the dermis. They are specialized in the synthesis of two types of protein fibers: collagen fibers and elastin fibers, constituents of the extra-cellular matrix. Collagen makes up 70% of the proteins of the dermis and provides it with its resistance to tensions and tractions.

The goal of this study is to evaluate the effect of the test element on the synthesis of collagen after 24-hour contact with a monolayer of normal human fibroblasts.

3. Test System

Cells Used:

The cells used are fibroblasts from a human child skin tissue (boy, Caucasian) on passage 2.

The fibroblasts are prepared and exposed according to the operating procedure in effect.

Reference Elements:

The following controls are included in each analysis:

Negative control: complete medium

Positive control: TGFβ 10 ng/mL

Definition of the Series:

Three non-cytotoxic dilutions of each sample, prepared in a culture medium with 0.005% of tween 60 and 0.05% of cotton oil, are tested in an amount of three wells per concentration.

The reference elements are also tested in three copies.

4. Course of the Study

Putting the Cells in Contact with the Product Under Investigation:

The cells are sown with 50,000 cells/cm². After 24 hours of culture, 37° C., 5% CO₂, they are incubated with the product under investigation and the reference element for 24 hours in a 1% FCS medium.

Assay of Collagen:

The collagen is assayed in the extra cellular matrix and in the culture medium by means of the assay box Sircol, Biocolor Ltd, Ireland, according to the procedure described in the box.

The assay is carried out with the red colouring agent Sirius (Direct Red 80) which has specific affinity for the trip helix structure (Gly-X-Y)ₙ of native collagen.

The absorbance of the dye-collagen complex is read out with the spectrophotometer at 540 nm.

A standard curve is produced between 0 and 50 μg/mL of collagen of type I.

Measurement of Cell Density:

The cell density is evaluated under the same conditions as previously, by measuring the activity of mitochondrial succinate dehydrogenase of living cells. This enzyme transforms MTT (3(4,5-dimethylthiazol-2-yl bromide)-2,5-diphenyltetrazolium) into blue formazan crystals. After dissolution of these crystals, a spectophotometric read out is performed. The measured absorbancies are proportional to the number of living cells.

5. Expression and Interpretation of the Results

In order to evaluate the cell density, the measured absorbance is expressed as MTT units (U MTT). It is proportional to the amount of cells present in each well.

The collagen concentration is expressed in pg of collagen /ml/U MTT in the extra cellular matrix and in the culture medium.

Results

| | Cell | Assay of collagen | | | Collagen ratio | | |
|---|---|---|---|---|---|---|---|
| | density | μg/wells (N = 3) | | | μg/wells/U MTT | | |
| Control N = 3 | Standard | Culture | Cell | | Culture | Cell | |
| Test item N = 3 | deviation | medium | matrix | TOTAL | medium | matrix | TOTAL |
| Negative | 0747 | 8.08 | 4.14 | 12.22 | 10.81 | 5.54 | 16.35 |
| control | 0.030 | 2.83 | 3.89 | | | | |
| Positive | 0.934 | 11.36 | 9.74 | 21.09 | 12.15 | 10.42 | 22.57 |
| control | | | | | | | |
| TGF β1 | 0.037 | 2.03 | 7.42 | | | | |
| (100 ng/ml) | | | | | | | |
| *Carrier | 0.834 | 9.58 | 3.13 | 12.70 | 11.48 | 3.75 | 15.23 |
| control | 0.014 | 1.41 | 6.10 | | | | |
| Pine PCO | 0.795 | 16.84 | 3.42 | 20.26 | 21.18 | 4.3 | 25.48 |
| esters | | | | | | | |
| 500 μg/ml | 0.053 | 3.92 | 3.61 | | | | |
| 200 μg/ml | 0.719 | 26.85 | 2.04 | 28.89 | 37.36 | 2.84 | 40.20 |
| | 0.062 | 9.00 | 7.68 | | | | |
| 50 μg/ml | 0.677 | 28.07 | 0.27 | 28.34 | 41.45 | 0.40 | 41.85 |
| | 0.032 | 5.55 | 1.92 | | | | |
| 5 μg/ml | 0.720 | 2.45 | 2.57 | 5.02 | 3.40 | 3.57 | 6.97 |
| | | 170 | 2.74 | | | | |

*carrier: a culture medium with 0.005% of tween 60 and 0.05% of cotton oil.

| | | Stimulation of | |
|---|---|---|---|
| | Stimulation of | collagen | Stimulation of the |
| | collagen excreted into | produced in | synthesis of total |
| | the culture medium | the cell matrix | collagen |
| Positive | 12% | 88% | 38% |
| control | | | |
| TGF β1 | | | |
| (100 ng/ml) | | | |
| Pine PCO | | | |
| esters | | | |
| 500 μg/ml | 84% | 15% | 67% |
| 200 μg/ml | 225% | 0% | 164% |
| 50 μg/ml | 261% | 0% | 175% |
| 5 μg/ml | 0% | 0% | 0% |

TGF β1 at 10 μg/mL significantly stimulates (38%) total synthesis of collagen. This result validates the model used. The stimulation is particularly effective at the cell matrix (88%).

As regards the effect of the product being tested, a very significant stimulation of the synthesis of total collagen may be noted with a maximum of more than 175% for a concentration of 50 μg/mL of the product.

It may also be observed that the product has an effect essentially on the secretion of collagen contrary to what may be observed for the positive control (TGF β1).

The effect is maximum for the concentrations of 200 and 50 μg/mL.

At 500 μg/mL, the effect remains highly pronounced but is less significant. At 5 μg/mL, the product no longer has any effect. This type of bell-shaped response is very often observed in this type of study.

6. Conclusion

Under the retained experimental conditions, pine PCO esters according to Example 1 have a substantial effect on the synthesis of collagen.

1-12. (canceled)

13. A treatment method for repairing the effects of skin ageing, comprising the application on the skin of a composition comprising:
a) procyanidolic oligomers (PCOs) extracted from pine barks, said oligomers being esterified with at least one fatty acid; and
b) a cosmetically acceptable carrier.

14. The method according to claim 13, for further preventing the effects of skin ageing.

15. The method according to claim 13, wherein the composition comprises:
a) a pine bark extract, the procyanidolic oligomers (PCOs) of which are esterified with at least one fatty acid; and
b) a cosmetically acceptable carrier.

16. The method according to claim 13, wherein the fatty acid is palmitic acid.

17. The method according to claim 13, wherein the composition is intended for topical use.

18. The method according to claim 17, wherein the composition is an ointment, cream, oil, milk, pomade, powder, impregnated pad, solution, gel, spray, lotion, suspension, soap or shampoo.

19. The method according to claim 15, where the composition comprises from 0.1% to 2.5% of the pine bark extract expressed by weight based on the total weight of the composition.

20. The method according to claim 13, for further soothing the skin.

21. The method according to of claim 13, for increasing the synthesis of collagen.

22. The method according to claim 13, for further protecting the skin against free radicals.
